(19) United States
(12) Patent Application Publication
(10) Pub. No.: US 2002/0015940 A1
(43) Pub. Date: Feb. 7, 2002

(54) Method of gene expression screening

(76) Inventors: Govind Rao, Columbia, MD (US); William E. Bentley, Annkcous, MD (US); Cornelia Renee Albano, New Market, MD (US)

Correspondence Address: SUGHRUE, MION, ZINN, MACPEAK & SEAS, PLLC, 2100 PENNSYLVANIA AVENUE, N.W., WASHINGTON, DC, 20037-3213, US

(21) Appl. No.: 09/813,315

(22) Filed: Mar. 21, 2001

Related U.S. Application Data
(60) Provisional application No. 60/193,036, filed on Mar. 29, 2000.

(51) Int. Cl. C12Q 1/00; C12Q 1/68; G06F 19/00; G01N 33/48; G01N 33/50; C12M 1/34

(52) U.S. Cl. 435/4; 435/6; 435/287.2; 702/20

(57) ABSTRACT

In a model system, Green fluorescent Protein fusions were constructed with several oxidative stress promoter probes from E. coli. These were chosen from the superoxide, hydrogen peroxide and hydroxyl radical inducible genes. When exposed to various free radical insults, the cells fluoresced with great specificity based on the corresponding regulon. These constructs are thus useful as a tool for mechanistic screening of a variety of anti-tumor drugs.

### CROSS-REFERENCE TO RELATED APPLICATION

This application claims benefit under 35 U.S.C. § 119(e) of Provisional Application Serial No. 60/193,036, filed Mar. 29, 2000, which is incorporated by reference herein, in its entirety.

### FIELD OF THE INVENTION

This invention is directed, inter alia, towards a method of using the oxidative stress response system in E. coli as a tool for mechanistic screening of anti-tumor drugs and other applications.

### BACKGROUND OF THE INVENTION

The current state-of-the-art in screening technology is primarily based on a molecular recognition event, typically a small molecule binding a target receptor/ligand or a nucleic acid sequence binding to a target. High Throughput Screening (HTS) technologies have been developed to allow for massive parallel processing. In particular, gene-chip technology has revolutionized the analysis of gene expression (Lockhart et al, Nature Biotechnol., 14:1675-1680 (1996); Gray et al, Science, 281:533-538 (1998); Wang et al, FEBS Letter, 445:269-273 (1999); Alon et al, Proc. Natl. Acad. Sci., USA, 96:6745-6750 (1999); and Zhu et al, Proc. Natl. Acad. Sci., USA, 95:14470-14475 (1998)). Here, thousands of DNA sequences of known genes are arrayed on a surface and the location of each is known. If one wishes to determine which genes are expressed in a sample, the mRNA from the sample is extracted, reverse transcribed to the corresponding cDNA, amplified, fluorescently labeled and allowed to hybridize with the sequences on the chip. Only the sequence-specific labels are captured on the surface of the chip. By reading the fluorescence, one can determine which of the genes were expressed.

While this technology is extremely effective and semiconductor fabrication technology has allowed for the packing of thousands of gene sequences into square centimeter surfaces at relatively low cost, there are a few limitations to its use. The actual experimental work involved is non-trivial, as one must first extract mRNA, convert the mRNA to cDNA, then amplify the cDNA, which then must be labeled for capture and detection. Each step is time and labor intensive and is not conducive for temporal studies involving a large number of samples. Furthermore, each of the steps involved in sample preparation for reading on a chip is prone to errors and artifacts.

For example, it is certain that the amount of mRNA does not necessarily correspond to the amount of final protein expression (Anderson et al, Electrophoresis, 19:1853-1861 (1998)). Secondly, converting mRNA to cDNA is potentially error prone with all mRNAs not being transcribed with equal efficiencies, which is known as reverse transcription bias. Any incomplete mRNA transcription will result in sequences that do not bind efficiently to their complements on the chip array. Additionally, specialized equipment is needed to produce and read the chip.

The present invention solves the above problems in that it can be easily executed by microbial culture and requires only generic equipment such as a plate reader, which is readily available in most labs, unlike a confocal gene-chip reader.

Anti-tumor drugs are typically screened using cell culture or mouse models. Only recently have genetic approaches towards screening been applied using simpler model species (Hartwell et al, Science, 278:1064-1068 (1997) ). The method of the present invention is similar to the SOS chromotest (Quillardet et al, Mut. Res., 147:65-78 (1985) ; and Quillardet et al, Proc. Natl. Acad. Sci., USA, 79:5971-5975 (1982)) and Ames test (Ames et al, Proc. Natl. Acad. Sci., USA, 70:782-786 (1973)) for mutagenicity, but offers additional specificity. The data resulting from screening in the method of the present invention can be used to interpret the in vivo mode of action of anti-tumor drugs and categorize them mechanistically. Additionally, the method of the present invention is adaptable to a high throughput-screening program for elucidating specific gene expression under a variety of conditions. In the present invention, various promoter probe-Green Fluorescent Protein fusions may be cultured in semisolid media in a multi-well plate and subjected to a battery of test compounds of varying concentration. In addition, several hues of Green Fluorescent Protein (GFP) are now available, and as a result, multiple genes within the same cell can be followed to elucidate temporal gene expression.

### SUMMARY OF THE INVENTION

In an embodiment of the present invention, known gene promoters are systemically cloned upstream of a readily measurable reporter gene and the gene expression is simply monitored by exposing the living cells to the perturbation (test compound). The advantage of such an approach is that the labor is shifted upstream of the experiment, i.e., to the creation of the clones. The experiments themselves become trivial by comparison, and are almost free of artifacts to which gene-chip experiments are subject.

As a model system, the examples herein use the oxidative stress response system in E. coli and show its applicability as a tool for mechanistic screening of anti-tumor drugs. The oxidative stress response of E. coli has previously been well characterized. In addition, many of the molecular mechanisms responsible for gene induction are understood and reflect high precision and sensitivity. It is shown in the present invention that specific genes are induced in response to reactive oxygen species such as superoxide anion, hydrogen peroxide and hydroxyl radicals and other DNA damaging (e.g. alkylating) agents.

FIG. 1 shows for the known genes of the E. coli oxidative stress response system and the regulons of which they are a part and to which active oxygen species they respond. In the examples herein, the delineated genes in FIG. 1 have been successfully cloned. The specific genes shown in FIG. 1 were deliberately selected from completely separate and different regulons in order to demonstrate that the present invention has the necessary specificity and generates a response only to stresses that are known to turn on only genes specific for each regulon. This demonstrates that the present invention has broad applications, i.e., it can be concluded with some confidence that if a gene is turned on, it is because of an exposure to a specific stress.

It is shown in the present invention that the unique fluorescence of GFP makes it a natural system for use as a reporter gene in the present invention. GFP is a relatively new reporter gene that is making an impact due to the many advantages it has over other reporter genes. These advantages include, but are not limited to: autofluorescence (Chalfie et al, Science, 263:802-805 (1994); and Prasher et al, Gene, 111:229-233 (1992)), in vivo detection (Chalfie et al, supra), no requirement for co-factors (Chalfie et al, supra), protein stability (Ward et al, Biochem., 21:4535-4540 (1982); and Bokman et al, Biochem. Biophys. Res. Comm., 101(4):1372-1380 (1981)), and availability of altered spectral mutants (Heim et al, Proc. Natl. Acad. sci., USA, 91:12501-12504 (1994); Delagave et al, Bio/Technol., 13:151-154 (1995); and Ehrig et al, FEBS Letter, 367:163-166 (1995)). GFP was originally discovered and isolated from the bioluminescent jellyfish Aequorea victoria (Shimomura et al, Aequorea J. Cell. Comp. Physiol., 59:223-239 (1962)). It absorbs light in the ultraviolet or blue range at 395 and 470 nm, respectively, and emits green fluorescence at 509 nm (Chalfie et al, supra) Recently, entirely new GFP families with additional colors have been reported (Matz et al, Nature Biotechnol., 17:969-973 (1999); and Mikhail, Nature Biotechnol., in press (1999)). However, it should noted that the present invention is not limited to use of GFP, and any reporter molecule can be employed in the present invention. Preferred reporter molecules include fluorescent reporter molecules, such as GFP, Yellow Fluorescent Protein, Red Fluorescent Protein and Cyan Fluorescent Protein.

GFP fluorescence has been shown to be quantitatively linked to the expression of a co-expressed heterologous protein (Albano et al, Biotechnol. Prog., 14:351-354 (1998)). The reporting abilities of GFP to those of the well-established chloramphenicol acetyl transferase (CAT) gene has also been compared. CAT is a bacterial gene that confers antibiotic resistance to chloramphenicol through acetylation. Its enzymatic activity can be assayed in a number of ways, making CAT a reliable reporter gene (Kain et al, In: Current Protocols in Molecular Biology, Ausubel et al, Ed., John Wiley & Sons, New York, NY, pages 9.6.1-9.6.12 (1995); and Rodriquez et al, Recombinant DNA Techniques: An Introduction, The Benjamin/Cummings Publishing Company, Inc., Menlo Park, Calif., pages 187-191 (1983)). In situ, GFP fluorescence was shown to be a measure of CAT activity and concentration when both GFP and CAT were expressed as an operon fusion (Albano et al (1998), supra). Using a number of proteins, it has been shown that GFP is a quantitative measure of the fusion product as extracted and assayed independently via both enzymatic analysis and Western blot (Albano et al (1998), supra; and Cha et al, Appl. Eviron. Microbiol., 65:409-414 (1999)).

In the present invention, after constructing cells containing stress-probe-GFP fusions, the cells were exposed to known oxidative stressors such as paraquat (a common name for methyl viologen), a known redox cycling generator of O₂ from O₂, or H₂O₂ for verifying the specificity of response. Unexpectedly, the GFP reporter gene allowed for a straightforward and rapid analysis of the oxidative stress response. In addition, the present invention only requires generic equipment such as a plate reader.

Pro-Tox© Assay, a related methodology developed by Xenometrix, Inc., utilizes 16 E. coli stress promoters fused to lacZ. The stress promoters used this assay are gyrA, katG, micE, osinY, uspA, katE, recA, zwf, dnaK, clpB, umuDC, merR, ada, dinD, soi28, and nfo. In the Pro-Tox© Assay, transfected cell lines are reconstituted from lyophilized vials and grown overnight. The optical density of the 16 cell lines are normalized to one another, aliquoted into the wells of a 96-well microtiter cell plate already containing additional media and are further incubated for 90 minutes.

A separate chemical plate containing 7 serial concentrations of the compound to be tested is prepared, aliquots are transferred to the cell plate and incubated for 90 minutes. A cell lysis reagent is then added to the cell plate and incubated for 15 minutes. The aliquots are then transferred to an assay plate containing the ONPG substrate and incubated for 10 to 30 minutes and read on a spectrophotometer plate reader at 420 nm. This entire procedure provides a single data point at the end, since the cells are lysed and destroyed.

In contrast, in the present invention one preferably starts with the growth of overnight cultures. Fresh cultures can then be prepared and grown for approximately 4 hours. Aliquots of these cultures can then be added to the wells of a microtiter cell plate containing the serial dilutions of the compound to be tested that are incubated overnight. The cell plates can then be read in a spectrofluorimeter at 509 nm. The two incubation steps, 4 hours and overnight, can be optimized. In addition, the cell plates can be read continuously to provide kinetic data on the actual rates of gene expression. A step-by-step comparison of the two methods is listed in Table I.

TABLE I

| | |
|---|---|
| Xenoanetrix Pro-Tox © Assay | GFP-Stress Probes |

| | | | |
|---|---|---|---|
| 1. | Begin with a Lyophilized | 1. | Begin with a |
| | culture | | frozen culture |
| 2. | Grown overnight | 2. | Grown overnight |
| 3. | Aliquot and dilute to | 3. | Transfer reinoculate |
| | microtiter plate | | (fresh media) |
| 4. | Incubate 90 minutes | 4. | Incubate 4 hours |
| | | | (may be changed) |
| 5. | Add compound to be tested | 5. | Aliquot to microtiter |
| | | | cell plate |
| 6. | Incubate 90 minutes | 6. | Add compound to be |
| | | | tested |
| 7. | Add lysis reagents | 7. | Incubate (can also be |
| | | | read continuously) |
| 8. | Incubate 15 minutes | 8. | Read at 509 nm |
| 9. | Transfer to assay plate | | |
| 10. | Incubate 10-30 minutes | | |
| 11. | Read at 420 nm | | |

Overall, the procedure of the present invention offers a clear advantage over the Xenometrix, Inc. Pro-Tox© Assay in that less steps are incurred in practicing the method of the present invention, thereby saving time and reducing chance of error when other reagents are added to the assay. In addition, the plates can be read continuously with the method of the present invention, whereas this does not occur with the Xenometrix assay due to lysis and destruction of cells to the point of providing a single data point at the end.

### BRIEF DESCRIPTION OF THE DRAWINGS

FIG. 1 shows the interplay between the reactive oxygen species, regulons in which they induce and the identified genes.

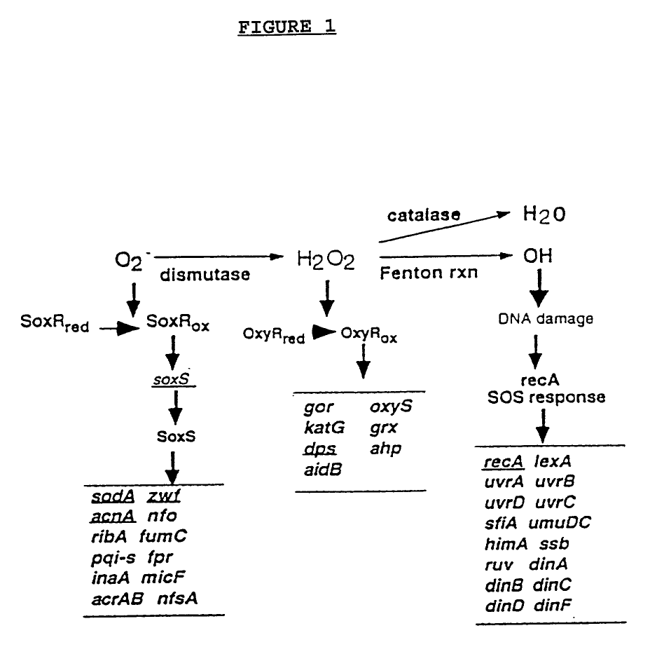

FIGS. 2A and 2B show the specificity of the response of various promoter probes to superoxide generating paraquat (PQ). neg is the negative control consisting of cells that express GFP based on an arabinose promoter.

FIGS. 3A and 3B show the specificity of the response of various promoter probes to hydrogen peroxide. neg is the negative control consisting of cells that express GFP based on an arabinose promoter.

FIG. 4 shows the induction of soda and recA promoter probes. A LB agar plate was overlaid with inoculated E. coli transformed with either the pCRAll, sodA promoter probe construct (FIG. 4A); or pCRA16, the recA promoter probe construct (FIG. 4B).

FIG. 5A shows the results of serially diluting Daunorubicin (DNR) for dose-response testing in a high throughput format; while FIG. 5B shows the results of serially diluting paraquat for dose-response testing in a high throughput format.

FIG. 6 compares the response of the sodA promoter to stresses in the form of a bolus of paraquat in one culture and transient exposure to hyperoxia in another. A control culture consisting of uninduced sodA:GFP is also included for comparison.

FIG. 7 illustrates a preferred embodiment of a system for screening for a compound that affects gene expression. The system comprises a computer 7.10 with a CPU and memory. A membrane 7.20 comprises an array, with each item in the array further comprising immobilized cell cultures and a test compound. 7.21 is an example of one such culture. Each culture, for example 7.21, is transformed with a DNA molecule encoding a reporter molecule fused to a promoter of a gene of interest, e.g., a GFP-promoter fusion. When the corresponding gene is expressed, the reporter molecule (GFP) is also expressed, resulting in a fluorescent spot. A light emitting diode 7.30, directs light onto a culture, thereby illuminating it. A photodiode 7.40 scans the illuminated culture and reads the fluorescent light emitted by the illuminated culture. An interface 7.50 receives signals from the photodiode and generates corresponding information for processing by the computer 7.10. The computer processes the information received from the interface to identify the gene whose expression was affected by the test compound.

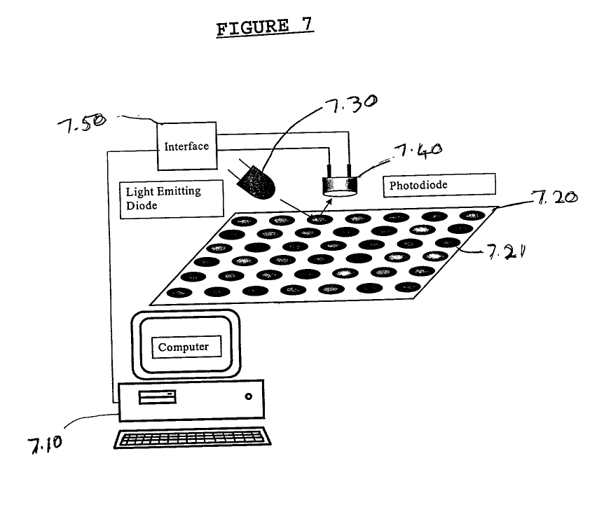

### DETAILED DESCRIPTION OF THE INVENTION

In order that the invention herein described may be more fully understood, the following detailed description is set forth.

“Regulon” as used herein refers to a set of genes that are all regulated by a common element or regulatory gene.

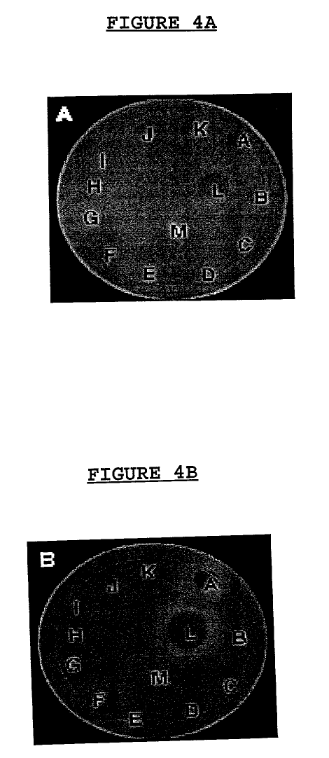

The GFP-stress probe fusions shown here clearly have great specificity and allow for the simple measurement of gene expression rapidly and continuously in vivo. Two promoter probes, containing the sodA: :gfp and recA: :gfp fusions, show the most striking results as shown in FIG. 4A and 4B. There appears to be a wide disparity in the effects the drugs have on the two promoter probes, which might be expected as they are from different regulons. sodA regulates superoxide dismutase production, while recA is a DNA repair protein that is presumably induced when hydroxyl radicals damage DNA.

Some drugs appear to have a very potent but limited induction range represented by a very narrow and bright emission of fluorescence. Other drugs appear to have a very large range of induction with the green fluorescence halo spread over a large area. FIGS. 4A and 4B show the sodA and recA plates that have been spotted at the same concentration of 500 μM for each drug. These panels can be used to compare the induction between drugs. As demonstrated and discussed earlier, the intensity of GFP fluorescence is quantitatively related to the level of gene expression (Albano et al, Biotech. Techniques, 10(12):953-958 (1996); and Albano et al (1998), supra). None of the anti-tumor drugs exceeded the ability of paraquat to induce the soda promoter. From this, the relative ability of drugs to induce a particular response in comparison to one another has been determined.

Many of the drugs induce both the recA and soda promoter probe suggesting the production of both the superoxide anion and the hydroxyl radical. This can be seen where higher and varied concentrations of drugs were used. In each case with one exception, the sodA promoter probe is induced to a lesser extent than recA. The sole exception is doxorubicin (K). Doxorubicin shows a stronger induction of sodA than its induction of recA.

Importantly, the microbial system of the present invention provides information relevant to mammalian cells. Three of the drugs examined, AZQ, DZQ, and MeDZQ, are structurally related to one another except for the substitution of side groups on the compound. These function as an alkylating agent that binds or cross-links DNA and also have the ability to generate superoxide anions, hydrogen peroxide, and hydroxyl radicals. Elsewhere, studies have been conducted to determine which of these two characteristics of the three drugs are responsible for the cytotoxicity in mammalian cells (Berardini et al, Biochem., 32:3306-3312 (1993); Lee et al, Biochem., 31:3019-3025 (1992); and Ngo et al, Chem. Res. in Toxicol., 11:360-368 (1998)). Consistent with these studies in mammalian cells, MeDZQ, DZQ, and AZQ, were ranked in decreasing order of toxicity response in the system of the present invention as well.

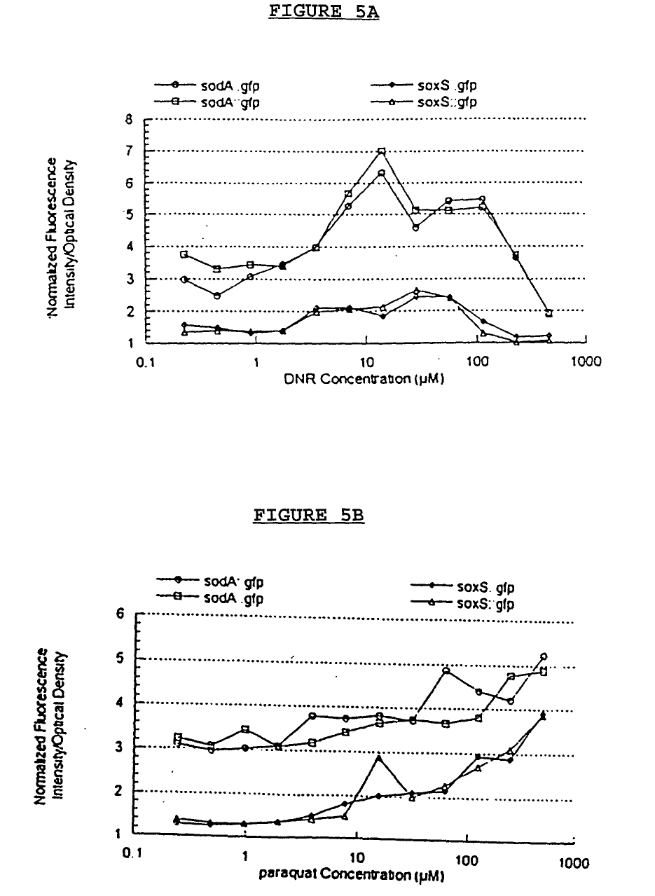

While the petri plate experiments initially carried out were encouraging, the data from such a format are not easy to quantify or use in a high throughput format. As noted from the high throughput format of the present invention, however, that DNR and paraquat have a similar mode of action, such as generating superoxide and turning on soxs and sodA, but also demonstrate different dose responses (See FIG. 5A and 5B). Maximal response is observed with 20 μM DNR after which it becomes toxic and kills cells, while paraquat continues to generate a monotonically increasing response. This indicates that the method of the present invention should allow for more precise investigation of the mode of action of anti-cancer drugs.

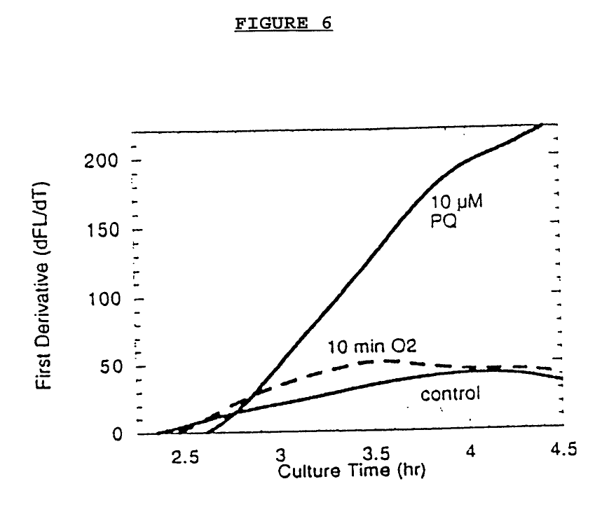

As the GFP derivative data in FIG. 6 demonstrate, continuous stress causes concomitant continual gene expression of sodA. In contrast, the short-term stress acts in a limited fashion where the rate of sodA expression reverts to the control value after an initial increase that is markedly lower than in the paraquat exposed cells.

The results herein demonstrate the further utility of GFP as a reporter gene for use in high throughput screening applications and in the creation of living chips. Here, an entire family of genes of known sequence but unknown function may be cloned upstream of a reporter gene, such as GFP. These cells could be plated at high density and exposed to a variety of conditions to elucidate their expression conditions. In combination with high sensitivity techniques such as fluorescence correlation microscopy, several thousand single cell clones can be placed on a chip. The availability of the entire human genome will require such a technology in order to elucidate the functions of the various gene sequences.

In order that the invention as described herein may be more fully understood, the following examples' are set forth. It should be understood that the following examples are for illustrative purposes only and are not to be construed as limiting the invention.

### EXAMPLE 1

### Construction of the Genes in the Oxidative Stress Response System

Using GenBank to obtain the genomic sequence for each of the oxidative stress response genes, PCR primers are designed to amplify the promoter region. Generally the target for amplification was approximately 300 base pairs and encompasses the sequence upstream from the native translational start site. The PCR primers had an approximately 18 base pair homology with the genomic sequence, included an appropriate endonuclease restriction digestion site and were flanked by 5 additional bases at each end. The entire promoter region flanked by incorporated restriction sites was cloned in frame with GFP on a plasmid. The stress probes were then transformed into E. coli.

### EXAMPLE 2

### Fluorescence Intensity After Oxidative Stresses

Cells containing each stress probe-GFP fusion were cultured to early log phase, stressed with varying concentrations of paraquat or H₂O₂, and the fluorescence intensity measured at 4 hours post-stress. Due to the lethality of the higher concentrations of stresses, dividing by optical density (OD) normalized the fluorescence intensity values.

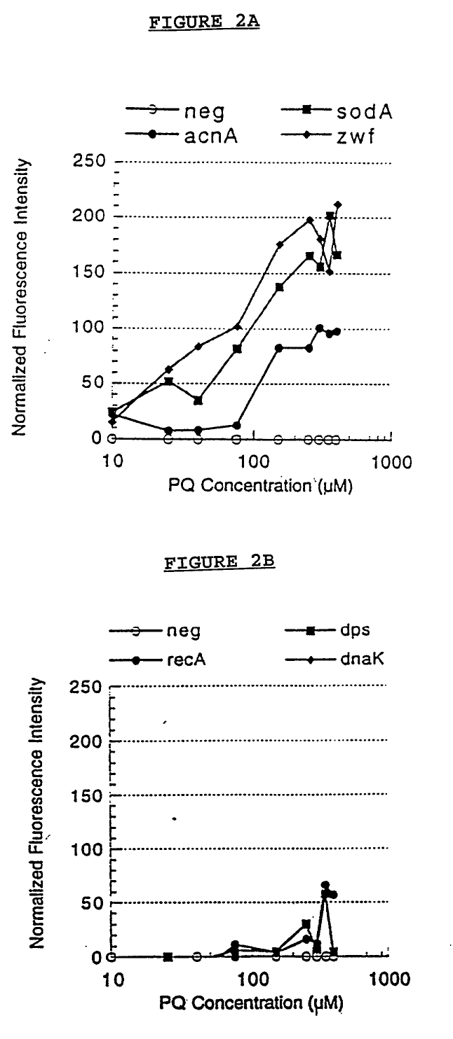

FIGS. 2A and 2B show the representative results of the promoter probes when stressed with paraquat. The sodA, zwf, and acna promoter probes, all belonging to the SoxRS regulon, show a dose dependent response (FIG. 2A). In contrast, the recA and dps promoter probes (FIG. 2B) do not show a dose dependent response and are only inducible when exposed to higher paraquat concentrations where presumably the greater 02 flux is converted to H₂O₂ and further exposed to the DNA damaging hydroxyl radical.

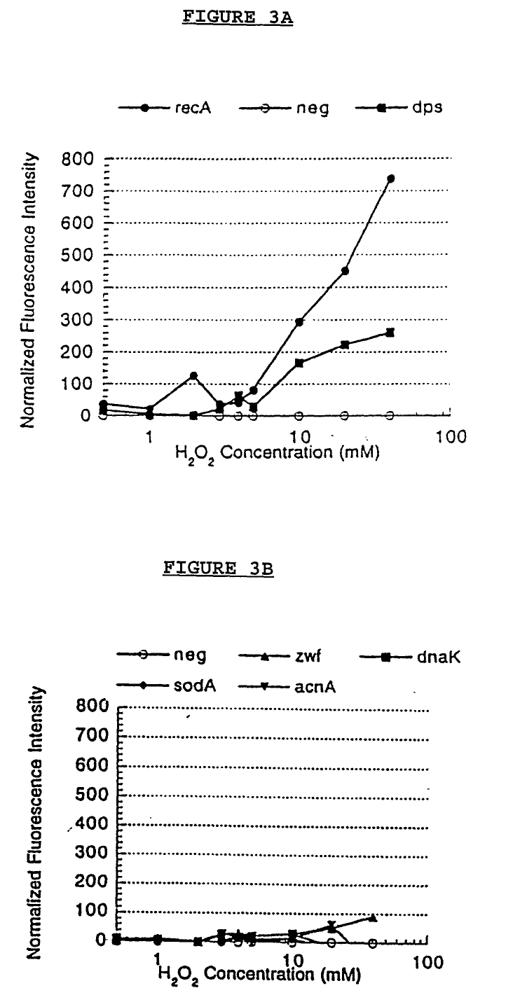

FIGS. 3A and 3B show the responses of each promoter probe when exposed to hydrogen peroxide. Hydrogen peroxide may cause DNA damage via hydroxyl radical formation. The recA and dps promoter probes shown in FIG. 3A show a dose dependent response to the hydrogen peroxide, while there is no inducible response seen in any of the SoxRS regulon promoter probes (sodA, acnA, and zwf) shown in FIG. 3B.

### EXAMPLE 3

### Mechanistic Studies with Anti-tumor Drugs

This Example involves the induction of sodA and recA promoter probes. A LB agar place was overlaid with inoculated E. coli transformed with oxidative promoter probe constructs. These probe constructs were either the pCRA11, sodA promoter probe construct (FIG. 4A); or pCRA16, the recA promoter probe construct (FIG. 4B). The desired transformants were selected by their resistance to ampicillin when plated on LB plates containing ampicillin.

2.0% cultures were grown from saturated overnight cultures at 350° C. with shaking at 260 rpm for 3 hours. A 4.0% inoculum was mixed with 550° C. top agar (10 g/L NaCl, 5.0 g/L yeast extract, 10 g/L tryptone, 7.0 g/L agar) and 10 ml was layered on 150 mm petri dishes containing LB and ampicillin. 10 μl of each drug to be tested was spotted onto the plate and incubated at 35° C. overnight. Colonies were screened when plated onto LB plates containing compounds that are known to activate oxidative stress genes, paraquat and hydrogen peroxide.

In FIG. 4A and 4B, spotting was carried out with each of the anti-tumor drugs at 500 μM concentration at different positions. The initial behind each anti-tumor drug description indicates its positioning in FIG. 4 and 4B. The anti-tumor drugs included: mitomycin C (MMC), A; streptonigrin, B; actinomycin-D,C; stretozotocin, D; diaziquone (AZQ), E; methyl diaziridinequinone (MeDZQ), F; paraquat (PQ), G; hydrogen peroxide, H; mitoxantrone, I; daunorubicin (DNR), J; doxombicin (ADR), K; cisplatin, L; and camptothecin, M.

Similarly, spotting was carried out with various concentrations of the anti-tumor drugs using the anti-tumor drugs as used above except their concentrations vary; MMC (1.5 mM), A; streptonigrin (1.0 mM), B; actinomycin-D (1.0 mM), C; streptozotocin (100 mM), D; diaziquinone (10 mM), E; methyl diaziridinequinone (12 mM), F; paraquat (100 mM), G; hydrogen peroxide (5.0 mM), H; mitoxantrone (10 mM), I; daunorubicin (5.0 mM), J; doxorubicin (100 mM), K; cisplatin (500 μM), L; and camptothecin (500 μM), M.

The plates were photographed on top of a longwave (365 nm) ultra-violet box with a Kodak DC200 digital camera, photographed while illuminated by a UV light box. In FIG. 4A and 4B, bright blue fluorescence was noted that is thought to be the autofluorescence of carnptothecin in the M position of each panel.

It was found herein that an E. coli based system could respond in a manner useful for interpreting data for drugs to be used in mammalian cells. Unexpectedly, the GFP reporter gene allowed for a straightforward and rapid analysis of the oxidative stress response. As the drug diffuses through the media, it created a concentration gradient. After overnight incubation, drugs may or may not show a zone of killing, and those capable of inducing the stress response promoter probe show a zone of induction that appears as a green halo when illuminated with a hand-held UV light.

The two promoter probes, containing the sodA: :gfp and recA: :gfp fusions, show the most striking results in FIG. 4A and 4B. There appears to be a wide disparity in the effects the drugs have on the two promoter probes, which might be expected as they are from different regulons. sodA regulates superoxide dismutase production, while recA is a DNA repair protein that is presumably induced when hydroxyl radicals damage DNA. Some drugs appear to have a very potent but limited induction range represented by a very narrow and bright emission of fluorescence. Others appear to have a very large range of induction with the green fluorescence halo spread over a large area.

FIG. 4A and 4B show the sodA and recA plates that have been spotted at the same drug concentration of 500 μM, and can be used to compare the induction between drugs. As demonstrated and discussed earlier, the intensity of GFP fluorescence is quantitatively related to the level of gene expression (Albano et al (1996), supra; and Albano et al (1998), supra) . None of the anti-tumor drugs exceeded the ability of paraquat to induce the sodA promoter. From this, the relative ability of drugs to induce a particular response in comparison to one, another can be determined.

Importantly, the microbial system of the present invention provides information relevant to mammalian cells. Three of the drugs examined, AZQ, DZQ, and MeDZQ, are structurally related to one another except for the substitution of side groups on the compound. These function as an alkylating agent that binds or cross-links DNA and these also have the ability to generate superoxide anions, hydrogen peroxide, and hydroxyl radicals. Elsewhere, studies have been conducted to determine which of these two characteristics of the three drugs are responsible for the cytotoxicity in mammalian cells (Berardini et al, supra; Lee et al, supra; and Ngo et al, supra) . Consistent with these studies from mammalian cells, MeDZQ, DZQ, and AZQ, were ranked in decreasing order of toxicity response in the system of the present invention as well.

### EXAMPLE 4

### Fluorescence Intensity and Optical Density

4. 0% cultures of E. coli harboring the stress probes were grown from a saturated overnight culture in 250 ml shake flasks containing 25 ml LB media and 100 μg/ml ampicillin at 35° C. with shaking at 260 rpm until the optical density reached 0.8 at 600 nm. 100 μl aliquots of culture were added to each well of a 96-well plate, along with 10 μl of various concentrations of the drug to be tested and incubated overnight. Experiments were run in duplicate. Fluorescence intensity and optical density measurements were taken using a Wallac 1420 VICTOR multilabel counter.

### EXAMPLE 5

### High Throughput Studies

To test whether the procedure of Example 4 would work in a quantitative high throughput format, an experiment was conducted where the anti-tumor drug DNR was serially diluted and dose-response tested in a 96-well plate. For comparison, a similar experiment was conducted with paraquat. FIGS. 5A and 5B show these results. As can be seen from the data, a rapid determination of an optimal dose response at the genetic level can be carried out. Experiments were conducted in duplicate and showed similar responses.

What is noteworthy is that DNR shown in FIG. 5A and paraquat shown in FIG. 5B have a similar mode of action in generating superoxide and turning on soxS and sodA, but the two also have different dose responses. Maximal response is observed with 20 μM DNR, after which it becomes toxic and kills cells, while paraquat continues to generate a monotonically increasing response. This demonstrates that the method of the present invention pertains to the mode of action of anti-cancer drugs.

### EXAMPLE 6

### Rate Measurement of GFP

The rate of change of the GFP signal measures the rate of the stress protein induction. This experiment compares the response of the sodA promoter to stresses in the form of a bolus of paraquat in one culture and a transient exposure to hyperoxia in another. A control culture consisting of uninduced sodA:GFP was included for comparison. FIG. 6 shows the rate of change of the GFP signal that measures the rate of the stress protein induction. FIG. 6 also shows the differential rate of gene expression when the culture containing the sodA-GFP stress probe was exposed to continuous stress by paraquat versus a 10 minute pulse of short-term stress by pure oxygen. An uninduced culture was included for comparison purposes.

### EXAMPLE 7

### Rate of Change of the GFP Fluorescence Measurement

The disadvantages of GFP as a reporter gene are its lag time for fluorescence (approximately 90 minutes) and extraordinary stability. One way to compensate for these is to measure the rate of change of the GFP fluorescence, which is possible using an LED-based sensor that permits continuous in situ measurements. The cell specific rate of change of GFP normalized to optical density is then a measure of the rate of gene expression of the stress protein.

While the invention has been described in detail and with reference to specific embodiments thereof, it will be apparent to one skilled in the art that various changes and modifications can be made therein without departing from the spirit and scope thereof.

What is claimed:

1. A method of screening for a compound that effects expression of a gene of interest comprising the steps of:
(A) preparing an array of transformed cell cultures each forming a loci in said array, wherein each transformed cell culture in said array comprises:
(i) a member of a first series of DNA molecules, wherein each member of said first series comprises a DNA sequence of a promoter of a different gene of interest upstream and adjacent to a DNA sequence encoding a reporter molecule, such that expression of said reporter molecule is controlled by said promoter; and
(ii) a member of a second series of DNA molecules, wherein each member of said second series comprises a DNA sequence encoding said different gene of interest, such that expression of a product of said different gene of interest is controlled by said promoter,
(B) exposing the transformed cell cultures in said array to a test compound;
(C) at each loci, assaying for a reporter characteristic of said reporter molecule; and
(D) identifying loci in said array which show a difference in said reporter characteristic relative to a control, and identifying the gene of interest present at said loci to thereby identify a test compound which effects expression of the gene of interest, wherein cells constituting said culture are not lysed during said method.

2. The method of claim 1, wherein said difference in said reporter characteristic relative to a control is a reduction of said reporter characteristic relative to said control, and thereby a test compound which inhibits expression of said gene of interest is identified.

3. The method of claim 1, wherein said difference in said reporter characteristic relative to a control is an increase of reporter characteristic relative to said control, and thereby a test compound which enhances expression of said gene of interest is identified.

4. The method of claim 1, wherein said cell cultures are bacterial cell cultures.

5. The method of claim 1, wherein each transformed cell culture comprises at least two members of said first series of DNA molecules, wherein each of said first series of DNA molecules encode a different reporter molecule; and at least two members of said second series of DNA molecules, wherein each of said second series of DNA molecules encode a different gene of interest.

6. The method of claim 1, wherein said reporter molecule is Green Fluorescent Protein (GFP), and said reporter characteristic is green fluorescence intensity.

7. The method of claim 1, wherein each gene of interest is a member of the E. coli oxidative response system.

8. The method of claim 7, wherein each gene of interest is selected from the group consisting of gyrA, katG, micF, osmY, uspA, katF, recA, zwf, dnaK, clpB, umuDC, merR, ada, dinD, soi28, sodA and nfo.

9. The method of claim 1, wherein cells are cultured in said array and said assaying is carried over the course of time so as to examine the effect of the resulting identified test compound on the rate of expression of said gene of interest.

10. The method of claim 5, wherein cells are cultured in said array and said assaying is carried over the course of time so as to examine the effect of the resulting identified test compound on the rate of expression of said gene of interest.

11. The method of claim 1, wherein said cells are cultured in said array and said assaying is carried over at an end point so as to examine the effect of the resulting identified test compound on expression of said gene of interest at said end point.

12. The method of claim 1, wherein said second series of DNA molecules is heterologous to said cell cultures.

13. The method of claim 1, wherein said second series of DNA molecules is native to said cell cultures.

14. The method of claim 2, wherein said gene of interest is a gene whose increased expression is associated with cancer, and said test compound is identified as an anti-cancer compound.

15. The method of claim 3, wherein said gene of interest is a gene whose reduced expression is associated with cancer, and wherein said test compound is identified as an anti-cancer compound.

16. A system for screening for a compound affecting gene expression, said system comprising:
a computer with a CPU and a memory;
a membrane comprising an array of cell cultures, said cell cultures comprising immobilized cells, each cell culture is a transformed cell culture which comprises:
(i) a member of a first series of DNA molecules, wherein each member of said first series comprises a DNA sequence of a promoter of a different gene of interest upstream and adjacent to a DNA sequence encoding a reporter molecule, such that expression of said reporter molecule is controlled by said promoter; and
(ii) a member of a second series of DNA molecules, wherein each member of said second series comprises a DNA sequence encoding said different gene of interest, such that expression of a product of said different gene of interest is controlled by said promoter;
a source of light adapted to emit light onto a selected cell culture from said array of cell cultures;
a photodiode adapted to scan a characteristic generated by the reporter molecule; and
an interface adapted to receive signals from the photodiode and provide information to the computer, wherein said computer is adapted to receive the information and perform said screening.
